(19) United States
(12) Patent Application Publication
(10) Pub. No.: US 2020/0315544 A1
(43) Pub. Date: Oct. 8, 2020

(54) SOUND INTERFERENCE ASSESSMENT IN A DIAGNOSTIC HEARING HEALTH SYSTEM AND METHOD FOR USE

(71) Applicant: dB Diagnostic Systems, Inc., Weston, CT (US)

(72) Inventor: Steven Brian Levine, Weston, CT (US)

(73) Assignee: dB Diagnostic Systems, Inc., Weston, CT (US)

(21) Appl. No.: 16/753,075

(22) PCT Filed: Sep. 24, 2018
(86) PCT No.: PCT/US18/52396

§ 371 Date: Apr. 2, 2020

Related U.S. Application Data
(60) Provisional application No. 62/569,171, filed on Oct. 6, 2017.

(51) Int. Cl. A61B 5/00; A61B 5/12; H04R 25/00; G10L 25/51

(52) U.S. Cl. CPC A61B 5/7221; A61B 5/123; A61B 5/126; A61B 5/6898; A61B 5/002; A61B 2560/0431; G10L 25/51; A61B 2560/0247; A61B 2560/0475; A61B 2562/0204; H04R 25/70

(57) ABSTRACT

A system that enables a hearing test administrator to reduce false indications of hearing loss in a patient and administer an interactive hearing assessment in a test environment is described. The system includes a sound measuring device to measure one or more background sounds of the test environment, and a data processor to process the background sounds, and determine the frequency and amplitude of the background sounds. The system may further include a memory that stores the backgrounds sounds, and its frequency and/or amplitude. The system may analyze whether the background sounds are above the maximum permissible ambient noise levels of the test environment and generate a report for analysis by the hearing test administrator.

### CROSS-REFERENCE TO RELATED APPLICATIONS

This application is a national phase application of and claims priority to Patent Cooperation Treaty (PCT) Application No. PCT/US2018/052396 filed Sep. 24, 2018, which claims priority to U.S. Provisional Application No. 62/569,171 filed Oct. 6, 2017, each of which is incorporated herein by reference in its entirety.

### FIELD

A system and method for reducing false indications of hearing loss in a patient is generally described. In particular, a system that enables a healthcare provider to reduce false indications of hearing loss in a patient administer interactive hearing assessments in a test environment is described.

### BACKGROUND

Incidences of hearing loss have been steadily increasing in the United States. In 1989, the reported incidence of hearing loss in the United States was 266 per thousand households, 283 per thousand households in 2004, and 295 per thousand households in 2008. In 2008, 43.25 million people reported hearing difficulty. Between 2004 and 2008, the hearing impaired population grew 8.8% compared to a 4.5% increase in US households. Furthermore, the percent of the population that acknowledged a hearing loss grew from 10% in 1989 to 11.3% in 2008.

People with hearing loss rarely acknowledge the disorder, which may lead to a large number of people remaining undiagnosed. From the time hearing loss is first noticed, the average hearing impaired person delays seven years before seeking assistance. Some reasons for the delay may include the perception by the hearing impaired that hearing aid devices geared toward treating hearing loss are expensive, thus they choose to not inquire about a hearing analysis. Others may fear ridicule and taunting by family and friends if they use hearing aid devices, and some others choose to avoid hearing assessments because acknowledging hearing loss may mean an acknowledgment that they are aging, and their bodies are unable to do things they once took for granted.

Technology used in hearing aids is improving rapidly in a manner parallel to other chip-based consumer goods. Newest technologies remain relatively expensive, but slightly older technologies are more affordable. Furthermore, continuous improvement has been made in designs to make hearing aids less cumbersome and noticeable in the ear, along with better ability to eliminate background and/or undesirable noises. Therefore, with early identification and the availability of improved treatment options, an increased number of hearing impaired individuals can benefit from hearing assistance.

Conventional hearing health assessment systems and methods often include a physician or other healthcare providers who perform hearing measurements/hearing tests, utilizing various devices to provide guidance on the matter. Such devices often serve to collect data that reflects the lowest sound level that a patient can hear (i.e., the patient's hearing threshold). In order to accurately collect the patient's hearing threshold, healthcare providers seek to conduct the hearing tests in a controlled environment, with reduced ambient sound and distractions to the patient. Efforts may include limiting sound and activities that may be perceived by the user and interfere with and/or reduce the accuracy of the hearing test. The source of such sound and activities may be located outside, but in the general area, of the test room. Others may be in the test room. While some medical offices may include rooms designed to attenuate noise external to the testing room, a soundproof test room is a fallacy. When hearing tests are conducted in these environments, they may reflect a high hearing threshold for the patient, which may result in a false indication of hearing loss (i.e., false positive hearing loss).

According to the disadvantages described above, there is a need for a system and a method that enables healthcare providers to identify the presence of ambient noise levels that may impact the result of a diagnostic hearing assessment. Further, there is a need for a system and a method that provides a simple, efficient, and cost-effective diagnostic hearing assessment, while enabling healthcare providers to assess whether their administration of interactive hearing assessments in test environments will result in a false indication of hearing loss in a patient.

### BRIEF DESCRIPTION

The present disclosure generally describes a system that enables a hearing test administrator to reduce false indications of hearing loss in a patient in a test environment. The system may also provide for the administration of a threshold hearing test, such as an interactive hearing assessment, in the test environment, as well as the gathering of relevant clinical history, and capturing of findings on relevant physical examination of the patient. According to an aspect, the system includes a sound measuring device to measure the background sounds of the test environment, which may be represented as a function of frequencies and amplitudes. Such background noise may indicate that ambient noise levels of the environment are above maximum permissible ambient noise levels of the test environment. The system may further include a memory that stores the background noise and/or the frequency and amplitude measured frequency ranges. It is contemplated that the system will generate a report that is reviewable by the hearing test administrator, so that the hearing test administrator can determine the risk of a false positive hearing loss result.

The present disclosure further describes the system including a hand-held, mobile hearing test device for the administration of a threshold hearing test by the healthcare provider in the test environment. The hearing test device transmits at least four distinct pure tone frequencies to the patient, and the patient activates a hand-held, mobile input device upon hearing the at least four distinct pure tone frequencies. The patient's activation of the hand-held, mobile input device indicates a result of the threshold hearing test. The hearing test administrator can assess the report and/or the hearing test results for indications of a false positive hearing loss, and whether the threshold hearing test needs to be re-administered and/or whether the hearing test needs to be conducted in another test environment.

The present embodiments also relate to a method of reducing false indications of hearing loss in a patient and administering an interactive hearing assessment in a test environment. The method includes capturing one or more background sounds of the test environment using the sound measuring device, and processing signals related to the background sounds to calculate a frequency and an amplitude of the background sounds. A data processor generates a report that includes the frequency and the amplitude, thus providing a hearing test administrator with data that enables the hearing health administrator to assess whether the background sounds may result in a false indication of hearing loss in the patient.

### BRIEF DESCRIPTION OF THE FIGURES

A more particular description will be rendered by reference to specific embodiments thereof that are illustrated in the appended drawings. Understanding that these drawings depict only typical embodiments thereof and are not therefore to be considered to be limiting of its scope, exemplary embodiments will be described and explained with additional specificity and detail through the use of the accompanying drawings in which:

FIG. 1 schematically illustrates a system that enables a hearing health administrator to reduce false indications of hearing loss in a patient when a threshold hearing test is conducted in a test environment, according to an aspect;

FIG. 2 schematically illustrates a system to enable a hearing health administrator to reduce false indications of hearing loss in a patient and administer an interactive hearing assessment of the patient in a test environment, according to an aspect; and

FIG. 3 is a flow chart illustrating a method of reducing false indications of hearing loss in a patient while administering an interactive hearing assessment in a test environment, according to an aspect.

Various features, aspects, and advantages of the embodiments will become more apparent from the following detailed description, along with the accompanying figures in which like numerals represent like components throughout the figures and text. The various described features are not necessarily drawn to scale, but are drawn to emphasize specific features relevant to some embodiments.

### DETAILED DESCRIPTION

Reference will now be made in detail to various embodiments. Each example is provided by way of explanation, and is not meant as a limitation and does not constitute a definition of all possible embodiments.

In an embodiment, a system, including various devices and/or mechanisms, and a method that enables a hearing test administrator to administer an interactive hearing assessment of a patient in a test environment is provided. The hearing test administrator may include any person who conducts a threshold hearing test, such as, for example healthcare providers. A “healthcare provider” is a provider of health related services that may or may not also be a hearing specialist, e.g., a primary care physician, internal medicine physician, family medicine physician, geriatric physician, gynecologist, audiologist, otolaryngologist, hearing-aid kiosk operator, neurologist, naturopath doctor, chiropractic doctor and the like.

As used herein, “ambient noise” refers to all encompassing noise normally associated with a given test environment (hearing test environment), including a composite of sounds within, near and far from the testing environment. Such testing environments may include, for example, an audiometric test room or any room where a hearing test is to be conducted.

As used herein, a “test environment” is the environment in which a hearing test is conducted. Typical test environments may include a soundproof testing room or booth, which mitigate the effect ambient noise levels (described in further detail hereinbelow) may have on the hearing test results. Other test environments include any non-soundproof testing room or medical office that is pervious to ambient noise levels. Hearing tests may be conducted using headphones that completely surround the ears of the patient (i.e., circumaural headphones), and serve to attenuate the unwanted ambient noise levels in the test environment. The unwanted ambient noise levels may exceed the maximum permissible ambient noise levels (MPANLs). The MPANLs, as specified by the American National Standards Institute (ANSI), are negligible ambient noise that would not mask pure tones during hearing assessments, as would be understood by one of ordinary skill in the art. An American National Standard published by ANSI, titled “Maximum Permissible Ambient Noise Levels for Audiometric Test Rooms”, and retrievable from www.whisperroom.com/pdf/ANSI-ASA-S3.1-1999-R2008.pdf is incorporated herein by reference in its entirety. The Standard specifies MPANLs from 125 Hz to 8,000 Hz, 250 Hz to 8,000 Hz and 500 Hz to 8,000 Hz, and is intended for use by all hearing test administrator, such as healthcare providers, who test hearing.

The system described herein is capable of providing a “hearing assessment”, an assessment of the person's sense of hearing that is not quite as rigorous as a traditional, full-blown hearing test performed by a hearing health specialist, but is sufficient to provide at least a baseline indication of hearing issues so that the patient can be referred to an hearing/ear specialist for further analysis. Such “hearing health specialist/(s)” are trained and qualified to conduct thorough evaluations of a patient's hearing (e.g. audiologist) or are trained in the medical and surgical management and treatment of the ear, nose, throat (ENT), and related structures of the head and neck (e.g., an otolaryngologist or ENT (Ear, Nose, Throat) doctor), any conduct hearing tests using specialized equipment, such as an audiometer.

By “interactive”, what is meant is of or pertaining to a two-way system of electronic communications by means of a computer. By “portable”, what is meant is capable of being easily carried or conveyed by hand.

As used herein, a “wired device” is a device that uses wires, cables or other means to connect to/communicate information with another device.

As used herein a “wireless device” is a device that uses Bluetooth® technology and/or a Wi-Fi network to communicate information with another device. Such devices may include tablets, phones, laptops, and the like. Such devices electronically transmit data pertaining to the patient from one location to another, using telecommunications technology, without requiring the use of cables, wires, and the like.

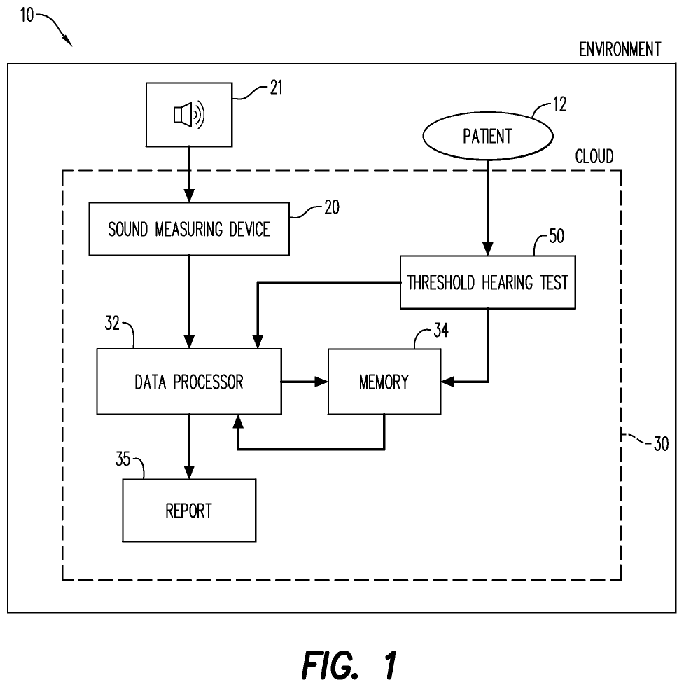

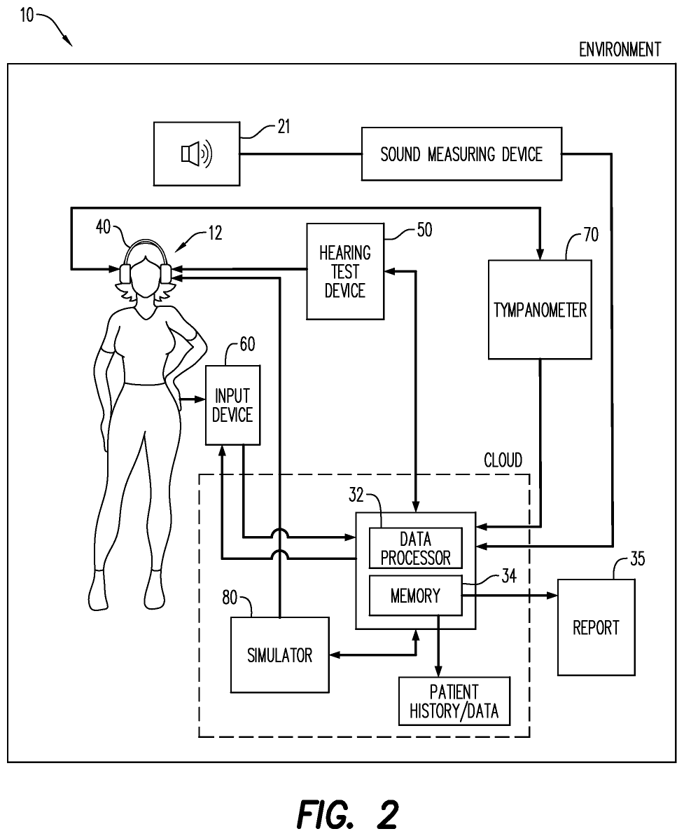

Now referring to FIGS. 1-2 and according to an embodiment, a machine, kit or system 10 (“system 10”) is described. The system 10 enables a hearing health specialist (not shown) to administer an interactive hearing assessment to a patient or test subject 12 in any environment, regardless of an ambient or background noise level. According to an aspect, the system 10 includes componentry for administering a portable hearing test as will be described in greater detail hereinbelow, and also componentry for measuring background ambient noise in the environment where the hearing test is being conducted. As shown herein, the system 10 includes several components/devices, each of which may be operable to administer the interactive hearing assessment and provide the healthcare provider with a report 35 including frequencies and amplitudes of background noise 21 detected from the test environment. Such components are illustrated as separate individual parts, although it would be understood by one of ordinary skill in the art that one or more of the devices/parts could be combined into a single device as described in more detail below.

A sound measuring device 20 is provided as a component of the system 10 for measuring ambient/background noise/sound 21 in the test environment and for transmitting the background sound detected to a data processor 32. The sound measuring device 20 may be housed in a device 30 and/or located in a network of servers and connections (the cloud 36) (described in further detail hereinbelow)). The sound measuring device 20 may include a microphone (not shown) to capture the background noise 21 of the test environment. The sound measuring device 20 is typically configured to measure sound pressure levels of the test environment, and the results of those measurements are reflected or represented in the system 10 as a function of frequency (i.e., pitch) and/or amplitude (i.e., loudness) of the background sound 21. The background noise 21 may include any number of daily life sounds of the test environment, such as, for example, 60 dB from a person speaking in or near the test environment, and any number of other background noises, such as lawn mowers, printers, fax machines, etc. The perceived loudness or magnitude of the background noise 21 may vary, thus the sound measuring device 20 captures any number of background noise 21 that may be above the MPANLs of the test environment to obtain a more accurate reflection of noise that may skew / negatively impact the patient's hearing test results. Exemplary devices capable of measuring the sound levels and/or frequency ranges include but are not limited to a sound level meter, integrated sound level meter, integrating-averaging sound level meter, sound pressure level (SPL) meter, sound dosimeter, decibel meter, and the like.

In an embodiment, the system 10 may include a microphone (not shown) to receive the background noise 21 of the environment, as will be described in further detail hereinbelow (see, for instance, FIG. 2). According to an aspect, when several different types of background noises 21 are captured by the sound measuring device 20, the data processor 32 identifies the frequency of each sound and may determine whether any of the identified frequencies are loud enough to potentially negatively impact or skew the patient's 12 hearing test results. The background noises 21, and the frequency and/or amplitude associated with the noises 21 may be stored in a memory 34. The hearing test administrator has access to the results of the background noise assessment stored in the system 10, and can access the results at any point during or after the patient's hearing test. The results serve to provide the hearing test administrator with information that alerts the administrator of the presence of ambient noise levels (i.e., noise levels above the MPANLs) that may result in a false indication of hearing loss for the patient 12. Thus, upon being alerted of such a risk, the hearing health administrator may decide whether to test the environment 2, 3, 4 or more times until the levels of the background noise 21 are below the MPANLs, identity and eliminate the source of any background noise 21 above the MPANLs, and/or to proceed with conducting the hearing test. Each test event may be stored in the memory 34, thereby allowing the test administrator to access each test event to determine whether there are any patterns of background noise 21. The hearing test administrator may use data pertaining to the test event/(s) to determine the circumstances of the test environment under which the levels of background noise 21 are typically below the MPANLs, and may proceed more confidently with knowledge of when it is best to conduct the patient's threshold hearing test. Thus, the hearing test administrator is provided with a tool that ultimately benefits the patient, by being able to provide the patient with an accurate diagnosis, without the risks associated with treating the patient for hearing loss when there is none. If the hearing test administrator properly determines and quantifies the level of the hearing loss in the patient, the hearing test administrator, such as a healthcare provider, will then be able to properly provide the patient with a plan for treating the patient 12 for any hearing health issues. This tool also provides benefits to the hearing test administrator, and diagnostic and treatment services in general, by reducing the rates of false indications of hearing loss in patients with healthy/normal hearing (i.e., hearing thresholds above 21dB, as would be understood by one of ordinary skill in the art).

The sound measuring device 20 may capture background sounds 21 of various frequencies. According to aspect, the sound measuring device measures background sounds 21 having frequencies up to about 10,000 Hz. In an embodiment, the sound measuring device 20 measures background sounds 21 having frequencies of from about 50 Hz to about 10,000 Hz, alternatively about 100 Hz to about 10,000 Hz, alternatively about 250 Hz to about 10,000 Hz. It is contemplated that the sound measuring device 20 may measure background sounds 21 at one or more sampling rates. According to an aspect, the sound measuring device 20 includes a software program that has an integrated sampling rate of 44,100 samples per second (i.e., 44.1 kHz). The samples may be gathered for a time period of from about 1 second to about 60 seconds, alternatively from about 10 seconds to 30 seconds, alternatively from about 10 seconds to 15 seconds. In an embodiment, the time period is manually selected by the hearing test administrator. Alternatively, the time period may be integrated within the software program, and the hearing test administrator may simply activate/select a “record” button/key/icon provided on the device 30. The integrated sampling rate and/or the sampling time period rate prevents and/or reduces the risk of the sound measuring device 20 capturing false background noises 21 (i.e., aliasing), along with accurately captured ones.

According to an aspect, the data processor 32 determines the amplitude (loudness) of the background noise 21 of the test environment. Data pertaining to the amplitude may be stored in the memory 34, and an average and/or median of multiple amplitudes are calculated by the system 10, so that variations/outliers of the loudness of the background noise 21 can be accounted for. Since the amplitude is a calculation of the forcefulness, loudness and/or intensity of the sound pressure level of the test environment, the greater the amplitude of the background noise 21, the greater the likelihood that the background noise 21 may skew the patient's hearing test results. This provides the hearing test administrator with an indication of whether the loudness of the sound in the environment is above the MPANLs, so that an assessment of whether results of hearing tests conducted in the test environment may be skewed can be made.

Once the sound measuring device 20 has gathered sufficient data (i.e., background noise 21), the data processor 32 notifies the hearing test administrator that the data is available for review and/or assessment. The data may be presented via a report 35 that includes the average and/or median levels of the one or more frequencies and the amplitude. It is contemplated that a recording of the background noise 21 may be provided in the report 35, and may be stored in the memory 34. The hearing test administrator may utilize the report 35 (with or without the recording of the background noise 21) in deciding whether to conduct the hearing test in a different test environment. In the event that the hearing test administrator proceeds with conducting the hearing test, the report 35 may also aid the hearing test administrator in making a determination of whether a diagnosis of hearing loss in the patient 12 is the appropriate action. In an embodiment, the report 35 includes a tabular display and/or a graphical display of the frequency and the median or average amplitude of the background sound 21. The report 35 may include a summary of the patient's data, including demographic information and health history, which may be inputted into the system 10 before conducting a hearing test or measuring the background noise 21 of the test environment (as will be described in further detail hereinbelow).

The system 10 may administer a portable interactive patient hearing assessment as generally described in U.S. application Ser. No. 14/190,924, filed Feb. 26, 2014, and U.S. Provisional Application No. 61/769,449, filed Feb. 26, 2013, each of which are incorporated herein by reference, in their entireties. The system 10 may further include a hand-held, mobile hearing test device 50 for administering the threshold hearing test. The hand-held, mobile hearing test device 50 is configured to transmit at least four distinct pure tone frequencies, at varying sound levels, to the patient 12, in a way that repeatedly measures a hearing threshold for each ear of the patient 12. The hearing test device 50 may be configured to transmit at least six distinct pure tone frequencies. The distinct pure tone frequencies may include 250 Hertz (Hz), 500 Hz, 1000 Hz, 2000 Hz, 4000 Hz and 8000 Hz. By “threshold hearing” what is meant is the methodology of providing varying levels of loudness to identify a level of hearing perceived by the patient 12. For instance, as a starting point, a higher threshold of sound is transmitted to the patient 12, and the patient 12 indicates that the sound has been received/perceived (as discussed in greater detail below). The threshold may thereafter be dropped to a level of loudness that would normally be considered too low for hearing, and the threshold is gradually raised until the patient 12 once again indicates that the sound has been heard. Then, the threshold is lowered once again, and gradually raised until the patient 12 once again indicates hearing. The threshold level is recorded accordingly to measure the level of hearing of the patient 12.

According to an aspect, the pure tone frequencies are sent to a headset 40 that is worn by the patient 12 (FIG. 2). The headset 40 may include a wired or Bluetooth® connection to the hand-held, mobile hearing test device 50. The headset 40 may or may not be a noise canceling headset. In an embodiment, the headset 40 is a Sennheiser HDA300 audiometer headphone.

In order to withstand rigor under US Food and Drug Administration procedures, and more importantly to qualify as an event that is reimbursable under personal health insurance plans, the hand-held, mobile hearing test device 50 should provide the hearing test in a repeatable, calibrated way. In other words, the hand-held, mobile hearing test device 50 may be calibrated and tested for calibration from time to time as necessary so that the various frequency tones and loudness of those tones are uniform across all devices. Accordingly, the hand-held, mobile hearing test device 50 may interact with a calibration device as necessary for calibration (not shown).

According to an aspect, the system 10 includes a hand-held, mobile input device 60. The hand-held, mobile input device 60 may request that the patient 12 respond to one or more questions designed to solicit background information about the patient 12. Such background information includes, but is not limited to, identifying demographic data and clinical history pertinent to issues surrounding the ear, hearing and balance, and may be a part of the patient's data (described hereinabove). The hand-held, mobile input device 60 may include, for example, a display (including e.g. a touch screen), a keyboard, a mouse or other pointing device, a switch/button, and componentry capable of providing output through the display, a speaker, and/or a printer, and is in communication with the data processor 32 (as described in more detail below).

According to an aspect, the hand-held, mobile input device 60 is configured for activation by the patient 12 upon the patient's hearing of the pure tone frequencies transmitted by the hand-held, mobile hearing test device 50. The patient 12 is able to respond to the lowest sound level they can identify by activating the hand-held, mobile input device 60. In other words, the patient 12 is able to respond to each pure tone frequency (i.e., the sound/(s) actually heard by the patient 12), by providing input to the hand-held, input device 60. According to an aspect, the hand-held, mobile input device 60 also transmits a signal that is indicative of the patient's response, to the data processor 32. The data processor 32 may be configured to process one or more signals indicative of the patient's response and issue signals associated with the hearing test based on the patient's ability to hear each of the at least four pure tone frequencies. The data processor 32 sends the hearing test result signals, which may include data that indicates hearing loss associated with the patient 12.

All components of the system 10, such as, the sound measuring device 20, the hand-held, mobile, hearing test device 50, and the handheld, mobile input device 60, may be integrated into a single unit/device 30; therefore, the data processor 32 and memory 34 may be included in the single unit 30. According to an aspect, the device 30 is a portable electronic device that is configured to host a plurality of modules/software incorporating the various functions (e.g., administering the hearing test, measuring the sound level, receiving patient input, and the like). The modules may form at least a portion of a platform for performing these functions, and may be downloadable onto the device 30, such as a personal or laptop computer, or onto any device, such as a hand-held mobile digital electronic portable device, a tablet, a telephone, or the like, whether or not such devices have internet access. As would be understood by one of ordinary skill in the art, activation of the hand-held, mobile input device 60 could be performed in many ways. In an embodiment, the hand-held, mobile input device 60 includes a switch/button that may be activated. In another embodiment, the patient 12 may touch and/or click on the screen of the device 30 (for instance, on the screen of a tablet, such as, Microsoft® Pro Surface, or iPad®). In the embodiment where a button is used, the button may be connected to a device that is wired to the hand-held, mobile input device 60 (not shown), as would be understood by one of ordinary skill in the art. Alternatively, the button may communicate wirelessly with the hand-held, mobile input device 60 (not shown), using a Bluetooth® or Wi-Fi connection.

In an embodiment, the threshold hearing test, the sound measuring device 20, the processor 32, the memory 34 may be provided in a platform made available to users via cloud computing. As used herein, the term “cloud computing” refers to when tasks are assigned to a combination of connections, software, and services, accessed over a network. The network of servers and connections is collectively known as “the cloud.” Computing at the scale of the cloud allows users to access supercomputer-level power from a portable electronic device 30 including all the individual components of the system 10. Using an access point like an iPhone® or iPad® device, available from Apple, Inc., an Android® tablet available from Samsung, a Windows® operating system available from Microsoft or a personal computer (PC) using Windows® operating system from any PC manufacturer, users can reach into the cloud for resources as they need them. It would be understood by one of ordinary skill in the art that software can be provided that is compatible with Android (an operating system based on Linux), Apple® iOS and Windows® operating systems as well so that the device 30 may be adapted for a variety of environments. Alternatively, various applications could simply be hosted or housed upon the device.

The system 10 may be used in conjunction with a tympanometer 70 configured to conduct a pressure test. The tympanometer 70 may be provided to conduct a middle ear compliance test in at least one ear of the patient 12. Tympanometry is an examination used to test the condition of the middle ear and mobility of the eardrum (tympanic membrane), and the conduction bones by creating an air pressure variation of the ear canal. The pressure test may serve as an objective means of testing the middle-ear functions, and thus, may be viewed in conjunction with the pure tone audiometry for an overall test of hearing. In evaluating hearing loss, tympanometry permits a distinction between the presence or absence of middle ear interference with respect to a measured hearing loss. Furthermore, in a primary care setting, a tympanometer 70 is typically useful for diagnosing otitis media by demonstrating the presence of a middle ear effusion. In any event, the tympanometer 70 is configured to transmit a signal indicative of the results of the pressure test to the processor 32. Any number of additional hearing test devices, such as, for example, a tuning fork, (not shown), may be used in conjunction with the system 10. The tuning fork may be particularly helpful in providing a preliminary assessment of unilateral hearing loss in the patient 12.

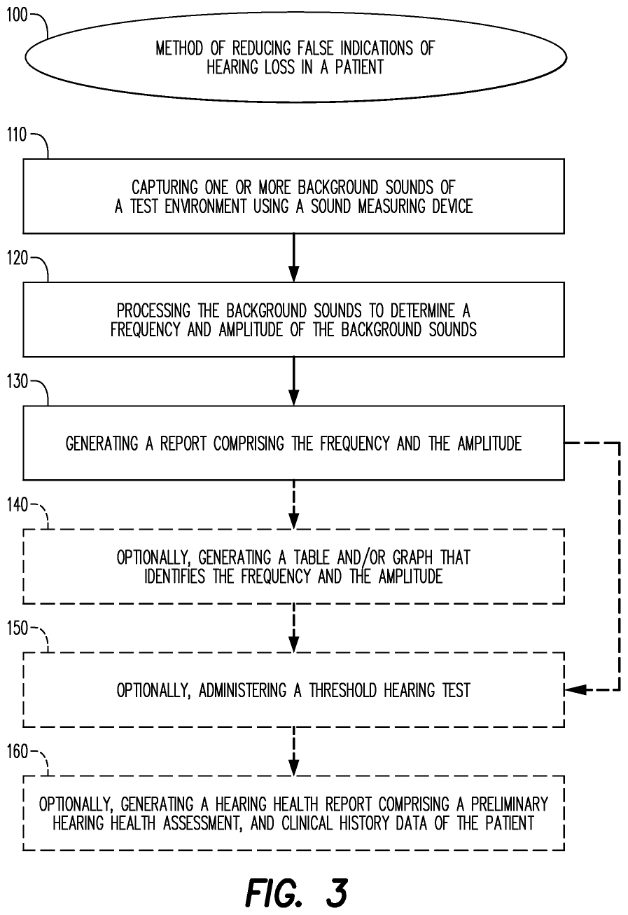

Embodiments of the disclosure further provide a method 100 of reducing false indications of hearing loss in a patient and administering an interactive hearing assessment in a test environment. As illustrated in FIG. 3, the method 100 includes capturing (i.e., measuring or recording) 110 one or more background sounds of the test environment using a sound measuring device, which typically includes a microphone. Signals related to the captured background sounds are processed 120 by a data processor, and the background noise is analyzed to determine a frequency and an amplitude of the captured background sounds. According to an aspect, the microphone and data processor are integrated within a hand-held mobile digital electronic portable device, such as a tablet.

The method further includes generating 130 a report based on the captured background sounds. The report may present the frequency and/or amplitude of the background sound in graphical or tabular displays that are easily reviewed by the hearing test administrator. According to an aspect, the report generates 140 the table/graph that identifies any sound levels that are above the MPANLs that may result in a false indication of hearing loss for the patient. The hearing test administrator can review this information and assess which, if any, sound may result in a false indication of hearing loss, if a threshold hearing test is conducted. In an embodiment, the system may optionally included suggestions of the potential source/(s) of the background sounds. The hearing test administrator may use these suggestions to identity and eliminate the source of any background noise 21 above the MPANLs, and proceed with conducting the hearing test. The report may be used to aid the hearing test administrator's decision on whether to further capture more background sounds of the test environment until sufficient data indicates that the levels of the background noise are below the MPANLs. If the report indicates that there are no background sounds or that the background sounds are below the MPANLs, the hearing test administrator may administer 150 the threshold hearing test, which may lead to more accurate hearing loss assessment. The threshold hearing test may be administered using the hand-held mobile digital electronic portable device (described hereinabove).

The components of the apparatus illustrated are not limited to the specific embodiments described herein, but rather, features illustrated or described as part of one embodiment can be used on or in conjunction with other embodiments to yield yet a further embodiment. It is intended that the apparatus include such modifications and variations. Further, steps described in the method may be utilized independently and separately from other steps described herein.

While the apparatus and method have been described with reference to specific embodiments, it will be understood by those skilled in the art that various changes may be made and equivalents may be substituted for elements thereof without departing from the scope contemplated. In addition, many modifications may be made to adapt a particular situation or material to the teachings found herein without departing from the essential scope thereof.

In this specification and the claims that follow, reference will be made to a number of terms that have the following meanings. The singular forms “a,” “an” and “the” include plural referents unless the context clearly dictates otherwise. Furthermore, references to “one embodiment”, “some embodiments”, “an embodiment” and the like are not intended to be interpreted as excluding the existence of additional embodiments that also incorporate the recited features. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Terms such as “first,” “second,” “upper,” “lower” etc. are used to identify one element from another, and unless otherwise specified are not meant to refer to a particular order or number of elements.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

As used in the claims, the word “comprises” and its grammatical variants logically also subtend and include phrases of varying and differing extent such as for example, but not limited thereto, “consisting essentially of” and “consisting of.” Where necessary, ranges have been supplied, and those ranges are inclusive of all sub-ranges therebetween. It is to be expected that variations in these ranges will suggest themselves to a practitioner having ordinary skill in the art and, where not already dedicated to the public, the appended claims should cover those variations.

Advances in science and technology may make equivalents and substitutions possible that are not now contemplated by reason of the imprecision of language; these variations should be covered by the appended claims. This written description uses examples to disclose the method, machine and computer-readable medium, including the best mode, and also to enable any person of ordinary skill in the art to practice these, including making and using any devices or systems and performing any incorporated methods. The patentable scope thereof is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal language of the claims.

1. A system comprising:
a sound measuring device for measuring one or more background sounds of a test environment;
a data processor for processing the background sounds, wherein the data processor analyzes the background sounds, and determines a frequency and an amplitude of the background sounds; and
a memory for storing the background sounds, wherein
the data processor generates a report comprising the frequency and the amplitude, and
the report provides a hearing test administrator with data that enables the hearing test administrator to assess whether the background sounds may result in a false indication of hearing loss in a patient undergoing a threshold hearing test.

2. The system of claim 1, wherein the amplitude is one of an average amplitude and a median amplitude.

3. The system of claim 1, further comprising:
a hand-held, mobile hearing test device for administration of the threshold hearing test by the hearing test administrator, wherein the hand-held, mobile hearing test device transmits at least four distinct pure tone frequencies to the patient; and
a hand-held, mobile input device for activation by the patient upon hearing the at least four distinct pure tone frequencies transmitted by the hand-held, mobile hearing test device, and for transmission of a signal indicative of the patient's response to the data processor.

4. The system of claim 1, further comprising:
a tympanometer configured to conduct a pressure test and to indicate the results of the pressure test.

5. The system of claim 1, wherein the sound measuring device is configured to measure background sounds having frequencies of from about 100 Hz to about 10,000 Hz.

6. The system of claim 1, wherein the sound measuring device is configured to measure background sounds at an integrated sampling rate of about 44,100 cycles per second.

7. The system of claim 3, wherein each of the sound measuring device, the hand-held, mobile hearing test device and the hand-held, mobile input device is integrated within a hand-held mobile digital electronic portable device.

8. The system of claim 1, wherein the sound measuring device notifies the test administrator when the background noise is above maximum permissible ambient noise levels of the test environment that may lead to false indications of hearing loss in the patient.

9. The system of claim 1, wherein the hand-held, mobile input device includes a portable electronic device that hosts a plurality of modules comprising the hearing test, the data processor and the memory.

10. A system for reducing false indications of hearing loss in a patient and administering an interactive hearing assessment of the patient's hearing in a test environment, the system comprising:
a sound measuring device for capturing one or more background sounds in the test environment, and determining a frequency and an amplitude of the background sounds;
a hand-held, mobile hearing test device for administration of a threshold hearing test in the test environment, wherein the hearing test device transmits at least four distinct pure tone frequencies to the patient; and
a hand-held, mobile input device for activation by the patient upon hearing the at least four distinct pure tone frequencies, wherein the activation indicates a result of the threshold hearing test.

11. The system of claim 10, further comprising:
a data processor for processing respective signals transmitted by the hand-held, mobile input device, and when the background sounds comprise a plurality of frequencies, calculating an average of the plurality of frequencies, and generating a report comprising the average of the plurality of frequencies; and
a memory to store the frequency or the average of the plurality of frequencies and the amplitude of the background sounds.

12. The system of claim 11, wherein the hand-held, mobile input device transmits one or more signals to the data processor, the signals being indicative of the patient's activation of the mobile input device.

13. The system of claim 11, wherein the data processor:
processes the one or more signals indicative of the patient's response;
issues one or more hearing test result signals associated with the hearing test based on the patient's ability to hear each of the at least four pure tone frequencies; and
sends the hearing test result signals indicative of hearing loss associated with the patient to the hand-held, mobile input device.

14. The system of claim 10, wherein the sound measuring device is communicably connected to the hand-held, mobile input device.

15. The system of claim 10, wherein the sound measuring device measures the background sounds having frequencies of up to about 10,000 Hz.

16. The system of claim 10, wherein the sound measuring device measures the background sounds at an integrated sampling rate of about 44,100 cycles per second.

17. A method of reducing false indications of hearing loss in a patient while administering an interactive hearing assessment in a test environment, the method comprising:
capturing one or more background sounds of the test environment using a sound measuring device;
processing the background sounds to determine a frequency and an amplitude of the background sounds; and
generating a report comprising the frequency and the amplitude, so that a hearing test administrator can assess whether the background sounds may interfere with a threshold hearing test conducted in the test environment and result in a false indication of hearing loss.

18. The method of claim 17, wherein if the background sounds comprise a plurality of frequencies and amplitudes, the method further comprises:
generating one of a table and a graph that identifies each frequency and amplitude, so that the hearing test administrator can assess whether a threshold hearing test conducted in the test environment will lead to false indications of hearing loss.

19. The method of claim 17, wherein the sound measuring device is integrated with a hand-held mobile digital electronic portable device.

20. The method of claim 17, further comprising:
generating a hearing health report comprising a preliminary hearing health assessment, and a clinical history data of the patient.
